(19) United States
(12) Patent Application Publication
(10) Pub. No.: US 2023/0268067 A1
(43) Pub. Date: Aug. 24, 2023

(54) A SMART, PERSONAL AND PORTABLE MEDICAL DIAGNOSTIC DEVICE

(71) Applicant: Swarna Kumari Adari, Visakhapatnam (IN)

(72) Inventor: Swarna Kumari Adari, Visakhapatnam (IN)

(21) Appl. No.: 18/005,254

(22) PCT Filed: Sep. 14, 2021
(86) PCT No.: PCT/IB2021/058374

§ 371 Date: Jan. 13, 2023

(30) Foreign Application Priority Data
Sep. 14, 2020 (IN) IN202041039807

(51) Int. Cl. G16H 40/63; B01L 3/00

(52) U.S. Cl. CPC G16H 40/63; B01L 3/50853; B01L 2300/0636; B01L 2300/023; B01L 2300/0861; B01L 2200/18

(57) ABSTRACT

A portable medical diagnostic device (100) to collect and analyze one or more biological specimen of a user is provided. The device (100) includes a housing having an interactive display (102) and a keypad (104). The keypad (104) comprises a plurality of selectable keys (106-1 or 106-2). The device (100) includes one or more biological specimen collection modules (108) operatively connected to the housing to receive at least one biological specimen of the user. One or more biological specimen collection modules (108) are communicably coupled with at least one selectable key of the selectable keys (106-1 or 106-2). The device (100) includes one or more sensors (110) provided in each of biological specimen collection module (108), configured to analyze at least one biological specimen received in the associated biological specimen collection module to determine one or more parameters of the biological specimen to be displayed on the interactive display (102).

### TECHNICAL FIELD

The present disclosure relates to a medical diagnostic device, and more specifically, to a personal and portable medical diagnostic device, having embedded sensors and Internet of Things (IOT) devices.

### BACKGROUND

Background description includes information that may be useful in understanding the present invention. It is not an admission that any of the information provided herein is prior art or relevant to the presently claimed invention, or that any publication specifically or implicitly referenced is prior art.

Peoples are curious about checking their health parameters regularly. For this, some people used to visit a hospital or a clinical or a path lab and some peoples used to have different electronics equipment at home.

Also, there is a need for regular check-up of health parameters such as but not limited to Diabetes: FBS, HbA1c, Kidney: BUN, Creatinine, BUN/Creatinine Ratio, Urine analysis, Electrolytes, Uric acid, Infection: CBC, ESR, Liver: Liver Functions Tests, HBsAg, Heart: Lipid Profile, Thyroid: T3, T4, TSH, Nerves: Vitamin B12, Anaemia: Iron, TIBC, UIBC, Transferrin Saturation %, Bone: Vitamin D, Calcium and many more.

However, recently due to spread of novel Corono Virus aka COVID-19, it is not possible or safe to visit health facilities for regular or routine check-up. The fear of COVID-19 is such that, people are not visiting any health facilities and avoiding regular check-up which are dangerous for elderly peoples and people who tends for caught infections regularly or people who are having low immunity.

Also, the use of medical specimen collection devices or systems which are used by every patient is not safe. No one knows the apparatus was sanitized properly after every use. It is similar to using a public toilet or utilities. We never know the person who used the public utilities before have some communicable disease or serious medical history.

Also, it is difficult to have all such medical instruments at home for self-testing. The cost of keeping every medical instrument for self-testing is too high and also, sometimes these medical instruments need precise knowledge for operation and procedure.

So, there is a need for a smart, personal and portable medical diagnostic device which is not costly, easy on operation and procedure. Also, to check the different samples of saliva, blood, urine at home without feeling the need to go any health facilities.

### SUMMARY OF THE INVENTION

It is a general object of the present disclosure for a smart, personal and portable medical diagnostic device for analysing one or more specimen or sample to be tested using sensors.

It is another object of the present disclosure for a smart, personal and portable medical diagnostic device for multiple specimen collection modules in each side of the device. In each module we can test different kinds of specimens with multiple Nano sensors and/or bio sensors embedded in each module or chemicals or any specimen identification chemicals.

It is another object of the present disclosure to provide a smart, personal and portable medical diagnostic device for printing and reporting the results to a communication device or a network printing device.

It is another object of the present disclosure to provide a smart, personal and portable medical diagnostic device for to share the result directly to one or more recipients selected from doctors, patients, and/or relatives of patients.

It is still another object of the present disclosure to provide a smart, personal and portable medical diagnostic device for generating results and/or analysing results to be stored in a local or remote database for permanent and/or temporary amount of time.

The smart, personal and portable medical diagnostic device comprises a touch screen display, power on-off button, microcontroller, RAM for storage of instructions and secondary memory for storage of results and user details, an imaging device, and a communication module.

The smart, personal and portable medical diagnostic device may include a rechargeable lithium-ion battery for providing power to the medical device and/or may operate with direct power supply to power on. The smart, personal and portable medical diagnostic device may also operate on Alkaline batteries incase of emergencies.

The smart, personal and portable medical diagnostic device also has a built in printer capability to print the test results then and there to share with the doctor/others.

If any module is designed for a specific specimen test then the system will show the information to the user while selecting the particular module to insert the correct specimen.

Once the correct specimen is inserted in the module and upon the specimen validation by the system then only it will start the specimen analyzing process else it will eject the module tray and ask the user to insert the correct/required specimen in the module.

The user or lab operator can also set the password to the device to avoid unauthorized access to the device.

The manufacturers can simply manufacture the modules to use by the users without buying the whole system again.

It is yet another object of the present disclosure to use the sensors, Artificial Intelligence and IOT (Internet of Things) technology in the smart, personal and portable medical diagnostic device.

### BRIEF DESCRIPTION OF THE DRAWINGS

The above and other objects, features and advantages of the present invention are further described in the detailed description which follows, with reference to the drawings by way of non-limiting exemplary embodiments of the present invention, wherein like reference numerals represent similar parts of the present invention throughout the several views, and wherein:

FIG. 1 is a block diagram of a portable medical diagnostic device;

FIG. 2 is an internal block diagram of the portable medical diagnostic device illustrated in FIG. 1;

FIG. 3 illustrates a hand-held extension portion of the portable medical diagnostic device;

FIG. 4 is a block diagram representing the components which may form part of an RF modem of the portable medical diagnostic device;

FIG. 5 is a block diagram representing the components which may form part of the extension portion;

FIG. 6 is a block diagram illustrating the components which form part of a local controller;

FIG. 7 is a block diagram illustrating the components which may form part of a host system;

FIG. 8A is a block diagram of the software hierarchy which may form part of a local controller processor;

FIG. 8B is a block diagram of the software hierarchy which may form part of an RF modem provided within a main portion;

### DETAILED DESCRIPTION

This above summary is provided to introduce a selection of concepts in a simplified form to be further described below in the Detailed Description. This summary is not intended to limit the scope of the claimed subject matter.

In order to overcome the above recited problems in the background section, the objective of the present invention is to provide a smart, personal and portable medical diagnostic device for collection of multiple specimens and a method that enables for automatic and real-time diagnose and analysis of a specimen without visiting any health facilities and/or using multiple independent medical devices.

The smart, personal and portable medical diagnostic device for multiple specimen collection modules in each side of the device. In each module we can test different kinds of specimens with multiple Nano sensors and/or bio sensors embedded in each module.

The smart, personal and portable medical diagnostic device comprises a touch screen display, power on-off button, microcontroller, RAM for storage of instructions and secondary memory for storage of results and user details, an imaging device, and a communication module.

The smart, personal and portable medical diagnostic device may also comprises biomarkers or bio-electronic chips as sensors.

The smart, personal and portable medical diagnostic device can connect to external devices such as breathe analyser, BP monitors, heartbeat monitor straps, pace makers, etc. . . .

The smart, personal and portable medical diagnostic device may include a rechargeable lithium-ion battery for providing power to the medical device and/or may operate with direct power supply to power on.

The smart, personal and portable medical diagnostic device also has a built in printer capability to print the test results then and there to share with the doctor/others.

Once the smart, personal and portable medical diagnostic device is switched on and the operating system which is proprietary is loaded. The interface of the smart, personal and portable medical diagnostic device then displays specimen icons on the display screen. These specimen icons relate to multiple health tests such as but not limited to blood test, urine test, skin test and saliva test.

The user may click or touch the specimen icon for the test on the screen. The selection of the test by the user by selecting the test icon the screen automatically and in real-time opens the corresponding module tray and ask to load the specimen into the dedicated slot.

The specimen is then submitted by the user module tray. The user then again clicks on the same icon so that the module tray will be closed automatically. The selection mechanism can be touch operated or may be physical buttons on the side of the device to operate.

In another embodiment, the user can pair his mobile computing device with the smart, personal and portable medical diagnostic device and send instructions only using the mobile computing device. The user can pair/connect the smart, personal and portable medical diagnostic device with the mobile computing device with any short communication technologies such as but not limited to Bluetooth, Infra-red, Wi-Fi, NFC etc.

The built-in microprocessor unit will process the specimen based on the given parameters and then the results will be display on the screen. The results can also be printed and sent to their devices through wired/wireless communication channels.

Each collection module pre-loaded with Nano/bio sensors and chemical compounds to examine the specimen. Once the sensors examined the specimen then the microprocessor will collate all the results and prepare a report and will be sending to external devices like mobile, printer, computer and monitor etc through wired or wireless communication.

The user can put multiple different specimens in different slots at the same time to examine the specimen. Each module in this device is independent to examine the specimen.

The smart, personal and portable medical diagnostic device can also save the profile of the user and also alerts the user for timely check-ups.

The smart, personal and portable medical diagnostic device can store the test results and compare the test results with the previous test to advise the user on improvement or non-improvement.

The smart, personal and portable medical diagnostic device also has internet connectivity to connect to the internet and suggest some treatment and advisory regarding the test based on the test results

The smart, personal and portable medical diagnostic device can also a physical or touch button to call the doctor. The contact information of the doctor or family member can be synchronized with the user mobile computing device.

Also, the contact information of related doctor or medical professional can be searched online and suggested.

The user can choose either to print the each specimen report or a complete report with all specimens submitted.

The firmware in this microcontroller can be upgraded to extend the capabilities of examining the different set of specimens or to include more tests.

This device also have dedicated button next to each module tray to open/close the specimen module tray manually.

Once the specimen is tested we can dispose the specimen collection module and insert a removable new collection module to examine another specimen. The specimen collection modules are bio-degradable and meant for single use.

Each specimen collection module has independent and unique printed electronic chips inside and pre-loaded with the required sensors to examine the specimen.

If any module is designed for a specific specimen test then the system will show the information to the user while selecting the particular module to insert the correct specimen.

Once the correct specimen is inserted in the module and upon the specimen validation by the system then only it will start the specimen analyzing process else it will eject the module tray and ask the user to insert the correct/required specimen in the module.

The user or lab operator can also set the password to the device to avoid unauthorized access to the device.

The manufacturers can simply manufacture the modules to use by the users without buying the whole system again.

The invention utilizes multiple Nano sensors, bio sensors, chemical compositions, IOT (Internet of Things), Artificial intelligence like technologies.

Referring now to the drawings in greater detail:

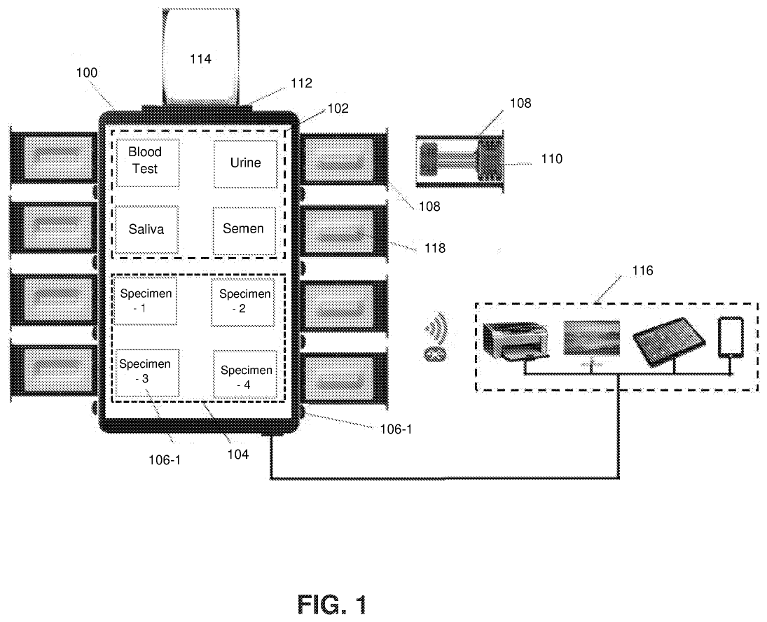

FIG. 1 is a block diagram of a portable medical diagnostic device. In an embodiment, a portable medical diagnostic device (100) to collect and analyze one or more biological specimen of a user is provided. The portable medical diagnostic device (100) includes a housing having an interactive display (102) and a keypad (104). The keypad (104) comprises a plurality of selectable keys (106-1 or 106-2).

The portable medical diagnostic device (100) includes one or more biological specimen collection modules (108) operatively connected to the housing to receive at least one biological specimen of the user. Each of the one or more biological specimen collection modules (108) are communicably coupled with at least one selectable key of the selectable keys (106-1 or 106-2).

Upon selection of the at least one selectable key, an associated biological specimen collection module from the one or more biological specimen collection modules is configured to emerge out the housing to receive the at least one biological specimen, and automatically get inserted back into the housing upon receipt of the at least one biological specimen.

The portable medical diagnostic device (100) includes one or more sensors (110) provided in each of the one or more biological specimen collection module (108). The one or more sensors (110) are configured to analyze the at least one biological specimen received in the associated biological specimen collection module to determine one or more parameters of the at least one biological specimen to be displayed on the interactive display (102).

In the embodiment, the housing encloses one or more electronic circuits to operate at least one of the interactive display (102), the keypad (104), the plurality of selectable keys (106-1 or 106-2), the one or more biological specimen collection modules (108), and the one or more sensors (110).

In the embodiment, each selectable key represents a unique operation, the unique operation selected from any one or a combination of emerging out the one or more biological specimen collection modules, getting the one or more biological specimen collection modules inserted back into the housing, analyzing the at least one biological specimen, and displaying the one or more determined parameters on the interactive display.

In the embodiment, the associated biological specimen collection module automatically gets inserted into the housing upon detecting presence of the at least one biological specimen and upon completion of a pre-defined interval of time.

In the embodiment, the one or more sensors (110) are selected from any one or a combination of a nanosensor and biological sensor.

In the embodiment, the housing encloses a processor or a microcontroller communicably coupled with the one or more sensors to process the one or more determined parameters by the one or more sensors to generate a printable report (114) causing a portable medical diagnostic device printer (112) embedded in the housing to print the printable report.

In the embodiment, the housing encloses a processor or a microcontroller communicably coupled with the one or more sensors to process the one or more determined parameters by the one or more sensors to generate a printable report. The processor is configured to transmit the printable report to one or more remote devices (116) through Internet of Things (IOT) devices embedded therein.

In the embodiment, each of the one or more biological specimen collection modules is loaded with one or more chemical compounds. The chemical compounds are used to treat or sample the specimens.

In the embodiment, each of the one or more biological specimen collection modules is removable, bio-degradable and replaceable.

In the embodiment, one or more biological specimen are selected from any one or a combination of serum samples, virology swab samples, biopsy and necropsy tissue, cerebrospinal fluid, whole blood for polymerase chain reaction (PCR), and urine samples.

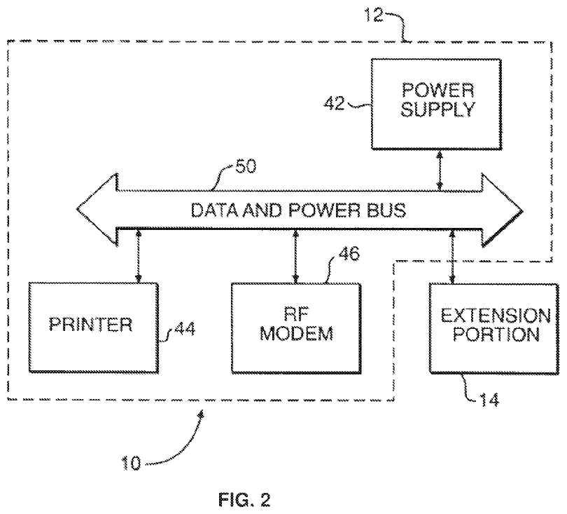

FIG. 2 is an internal block diagram of the portable medical diagnostic device illustrated in FIG. 1. Main portion 12 of the illustrated portable medical diagnostic device 100 is provided with a number of elements coupled to each other by means of a common data and power bus 50. Main portion 12 includes a power supply 42, a printer 44 and an RF modem 46. Extension portion 14 is also connected to data and power bus 50.

Power supply 42 is connected to each of the devices 44, 46 and 14 via data and power bus 50 in order to supply power (from a DC battery) to those devices. Data and power bus 50 also provides a communication path to facilitate the transfer of data between each of the devices. For example, information to be printed on a receipt may be forwarded by extension portion 14 to RF modem 46 over data and power bus 50. RF modem 46 may then transmit through data and power bus 50 sales data to printer 44 for printing of a receipt.

In the illustrated embodiment, each device, including printer 44, RF modem 46, and extension portion 14 has its own housing, and power supply 42 is housed within one of the main portion devices, i.e., it is housed within either printer 44 or RF modem 46. Power supply 42 may be provided as a separately-housed component of main portion 12, or even as a component of extension portion 14. However, in order to reduce the weight of hand-held portion 14, power supply 42 may be provided as part of main portion 12. More specifically, it may be provided within the housing of either printer 44 or RF modem 46. A DC converter may be provided as part of data and power bus 50 to facilitate the proper coupling of power to the other devices within main portion 12 and extension portion 14.

Portable RF modem 46 may be implemented with a standard off-the-shelf component. Specifically, portable RF modem 46 may comprise an RF modem model no. RFM 3010-000501-1 provided by Symbol Technology.

Printer 44 may comprise a Comtec RP3 Journal thermal printer provided by Comtec Info Systems, 30 Plan Way, Warwick, R.I., 02886. The RP3 Comtec thermal printer is provided with a power supply which may comprise power supply (not shown in FIG. 1). Printer 44 may include an internal program that interprets data sent to it in order to control the printing process. The interface to the program provided within the RP3 printer is described in the RP3 Receipt Printer User's Manual, Revision 60, Jan. 18, 1995, the content of which is hereby incorporated by reference herein in its entirety.

Extension portion 14 may comprise a Data Card 290e pin pad, which is a standard pin pad provided by Data Card Corporation, 11111 Bren Road, Minnetonka, Minn. 55343. Should extension portion 14 be implemented with a 290e pin pad provided by Data Card, the pin pad interprets data sent to it and performs requested actions in accordance with a protocol as defined in the Multiple Emulation PIN PAD (MEPPA) Reference Manual (Part No. 144214-001, Revision 1.10, October 1995), the content of which is hereby expressly incorporated by reference herein in its entirety.

Each of the devices including printer 44, power supply 42, portable RF modem 46, and extension portion 14 may be interconnected with the use of a custom designed cable unit, part no. 50-16000-106 provided by Symbol Technology, one Symbol Plaza, Holtsville, N.Y. 11742-1300.

Main portion 12 performs several main functions including two-way communications to and from off-site host 36 via an RF antenna 24, processing of sale transactions, and communicating with extension portion 14 via a data interface provided within main portion 12. Extension portion 14 performs functions including reading financial account data from an account authorization device provided by a customer, inputting a security code access key (e.g., a PIN) or user identification (ID), and generating encrypted data representative of the security code access key before such data is forwarded to main portion 12. The encrypted data representing the security code access key is subsequently forwarded to off-site host 36 which verifies the account and authorizes (or denies) access to the account.

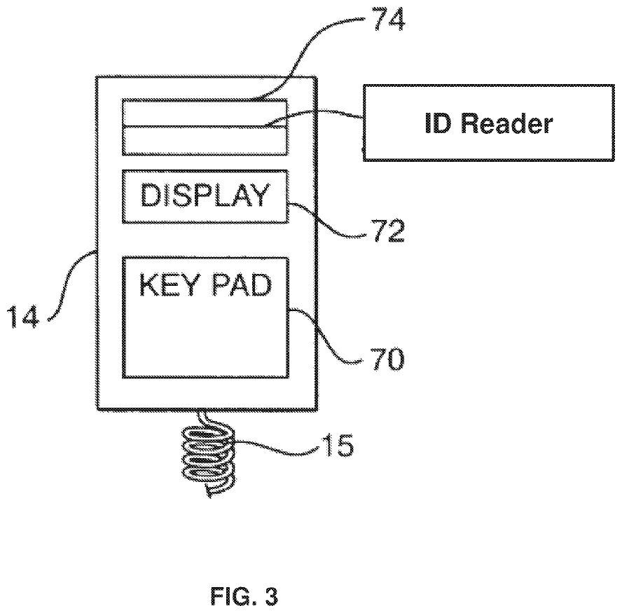

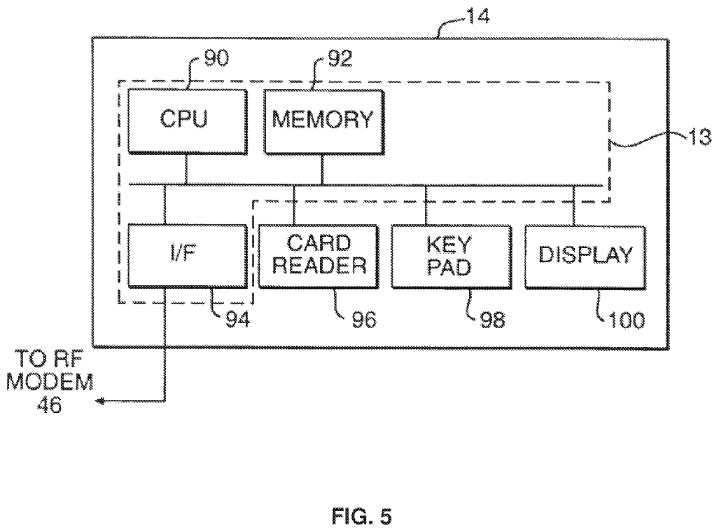

FIG. 3 illustrates a hand-held extension portion of the portable medical diagnostic device. As shown in FIG. 3, extension portion 14 may be provided with a magnetic strip reader 74, a display 72, and a keypad 70. FIG. 5 also illustrates an exemplary cord 15 in more detail. The cord 15 illustrated in FIG. 3 is in the form of a coil generally similar to the cord which is typically used to attach a telephone handset to a main telephone housing.

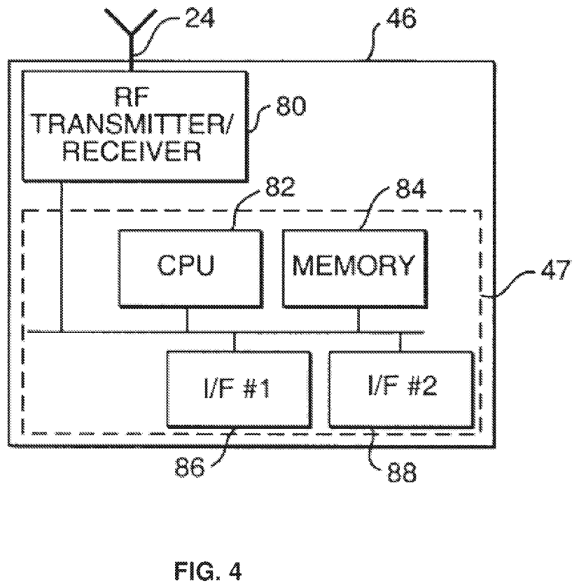

FIG. 4 is a block diagram representing the components which may form part of an RF modem of the portable medical diagnostic device. As shown in FIG. 4, RF modem 46 includes an RF transmitter/receiver 80 coupled to a remote RF antenna 24, and further coupled to a bus structure. The bus structure interconnects a main portion CPU 82, a main portion memory 84 (which may comprise a RAM), a first main portion interface 86, and a second main portion interface 88. The bus structure shown in FIG. 4 is merely illustrative, and may take the form of any recognized bus or interconnection structure which facilitates the interconnection of the various components of main portion 12. First main portion interface 86 is provided for connecting main portion 12 to extension portion 14, and second main portion interface 88 is provided for connecting main portion 12 to printer 44.

FIG. 5 is a block diagram representing the components which may form part of the extension portion. As shown in FIG. 5, extension portion 14 may include an extension portion CPU 90 connected to several other components via a bus structure, including an extension portion memory 92, and an extension portion interface 94. Other devices connected to the bus structure include card reader 96, keypad 98 and display 100.

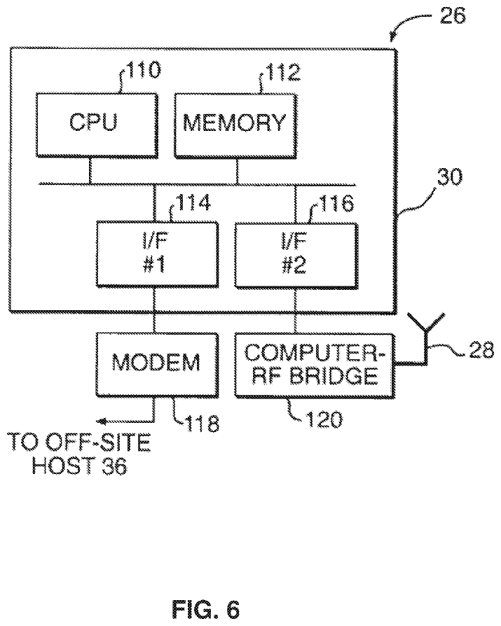

FIG. 6 is a block diagram illustrating the components which form part of a local controller. FIG. 6 is a block diagram illustrating a local controller 26, which is implemented with the use of a local computer 30. Local computer 30 comprises a number of devices, coupled to each other by a bus structure. Those devices include, among other devices, a local controller CPU 110, a local controller memory 112, and first and second local controller interfaces 114, 116. Local controller interfaces 114 and 116 may comprise serial ports, or other suitable hardware communications interfaces. A modem 118 is connected to first local controller interface 114 and provides a connection between local controller portion 26 and off-site host 36. A computer-to-RF bridge 120 is connected to second local controller interface 116, and includes a local RF antenna 28 which forms part of an RF channel for communicating with each of the respective portable device 100, e.g., as illustrated in FIG. 1.

Local computer 30 may comprise an IBM compatible PC, and computer-to-RF bridge 32 may be implemented with a Symbol Technologies serial access bridge SAB, Lite (SAB 2070-050) with a power adaptor. Local RF antenna 28 may comprise a Symbol Technologies Sandra D antenna. The interfaces provided as part of portable RF modem 46 may comprise serial interfaces, such as the serial interfaces which are provided in an off-the-shelf Symbol Technologies RF modem part no. RFM3010.

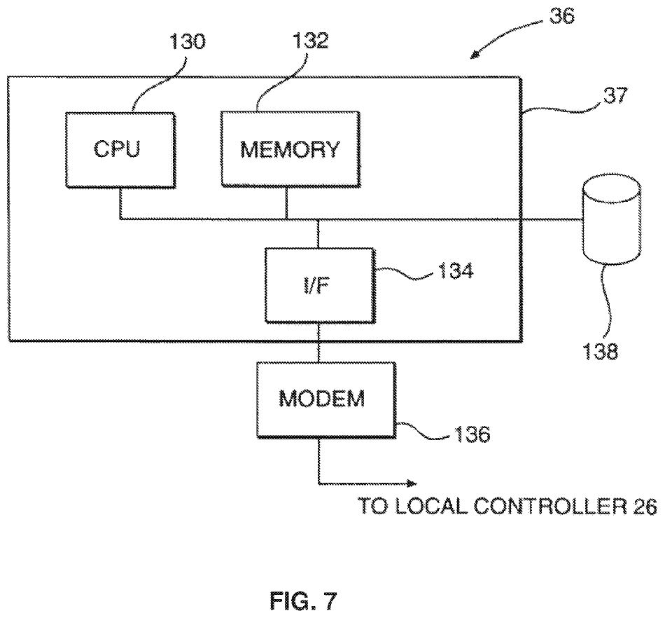

FIG. 7 is a block diagram illustrating the components which may form part of an off-site host 36. Off-site host 36 may comprise a host computer system which includes several devices connected to each other via a bus structure. The details of the bus structure are not disclosed herein, as such details are not directly pertinent to the present invention. The illustrated off-site host 36 includes a host CPU 130, a host memory 132, and a host interface 134. A modem 136 is connected to host interface 134, and provides connection of host 36 to local controller 26. Host 36 may further comprise a long-term storage 138, e.g., magnetic or magneto-optic disk or serial tape storage devices.

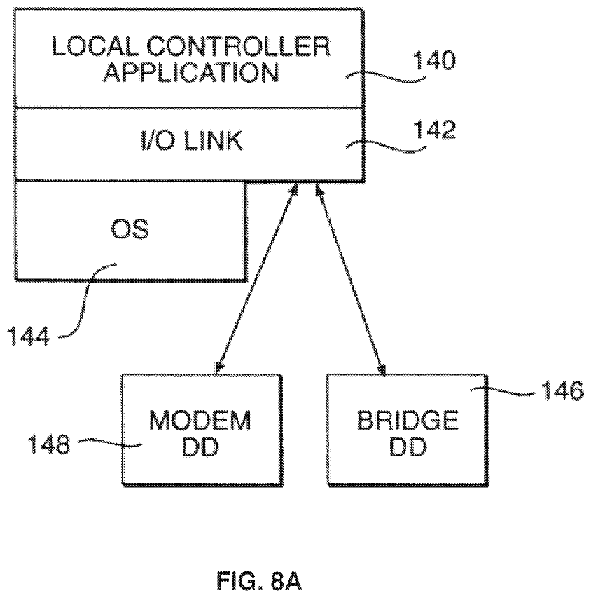

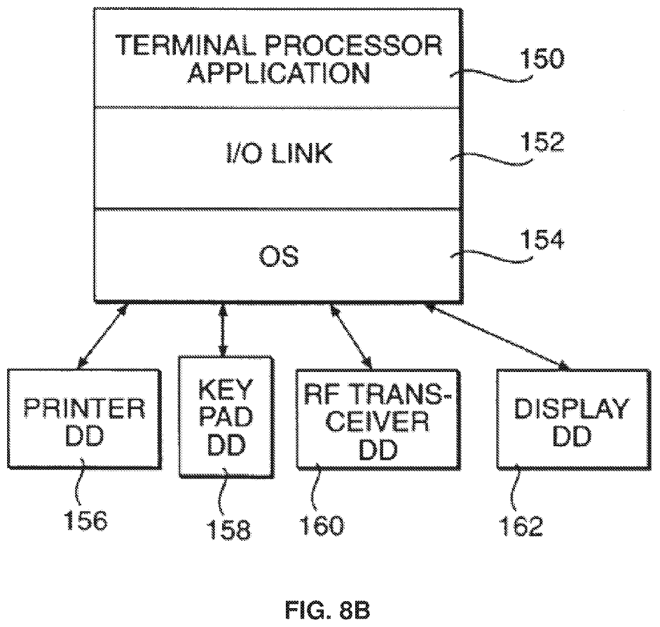

FIG. 8A is a block diagram of the software hierarchy which may form part of a local controller processor. FIG. 8A is a block diagram illustrating the basic hierarchy of the software that may be utilized to control the operation of local controller CPU 110 of local controller 26. The software hierarchy includes an operating system layer 144 provided at a lower level and an i/o link layer 142 provided at a middle level. A high-level software layer is provided which comprises a local controller application layer 140. The software of local controller 26 may comprise a plurality of device drivers, including a modem device driver 148 and a computer-to-RF bridge device driver 146, each directly coupled to i/o link layer 142.

FIG. 8B is a block diagram of the software hierarchy which may form part of an RF modem provided within a main portion. FIG. 8B is a block diagram illustrating the basic hierarchy of the software which may be used to control the operation of RF modem CPU 82 which forms part of RF modem 46 as illustrated in FIG. 4. The illustrated exemplary software hierarchy includes an operating system layer 154 provided at a lower level and an I/O link layer 152 provided at a middle level. A terminal processor application layer 150 is provided at a high level. A plurality of device drivers may be provided, each coupled to operating system layer 154. In the alternative, each of the device drivers may be coupled directly to either I/O link layer 152 or terminal processor application layer 150. The illustrated device drivers include a printer device driver 156, a key pad device driver 158, an RF transceiver device driver 160 and a display device driver 162. Key pad device driver 158 and display device driver 162 are provided to facilitate the input and output of data respectively from and (referring back to FIG. 7) key pad 98 and display 100 provided within extension portion 14 of portable POS terminal 10. Printer device driver 156 and RF transceiver device driver 160 are provided to facilitate the input and output of data respectively from and to (referring back to FIG. 2) printer 44 and RF modem 46 provided within main portion 12. While these functions are controlled by separate device drivers as shown in FIG. 10B, one or more unitary device drivers may be provided for controlling the interfacing and transfer of data to and from plural sets of the hardware devices, rather than providing a separate device driver for each hardware device.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

I claim:

1. A portable medical diagnostic device (100) to collect and analyze one or more biological specimen of a user, the portable medical diagnostic device (100) comprising:
a housing having an interactive display (102) and a keypad (104), wherein the keypad (104) comprises a plurality of selectable keys (106-1 or 106-2);
one or more biological specimen collection modules (108) operatively connected to the housing to receive at least one biological specimen of the user,
wherein each of the one or more biological specimen collection modules (108) are communicably coupled with at least one selectable key of the selectable keys (106-1 or 106-2), and
wherein upon selection of the at least one selectable key, an associated biological specimen collection module from the one or more biological specimen collection modules is configured to:
emerge out the housing to receive the at least one biological specimen, and automatically get inserted back into the housing upon receipt of the at least one biological specimen;
one or more sensors (110) provided in each of the one or more biological specimen collection module (108), the one or more sensors (110) are configured to analyze the at least one biological specimen received in the associated biological specimen collection module to determine one or more parameters of the at least one biological specimen to be displayed on the interactive display (102).

2. The portable medical diagnostic device as claimed in claim 1, wherein the housing encloses one or more electronic circuits to operate at least one of the interactive display (102), the keypad (104), the plurality of selectable keys (106-1 or 106-2), the one or more biological specimen collection modules (108), and the one or more sensors (110).

3. The portable medical diagnostic device as claimed in claim 1, wherein each selectable key represents a unique operation, the unique operation selected from any one or a combination of emerging out the one or more biological specimen collection modules, getting the one or more biological specimen collection modules inserted back into the housing, analyzing the at least one biological specimen, and displaying the one or more determined parameters on the interactive display.

4. The portable medical diagnostic device as claimed in claim 1, wherein the associated biological specimen collection module automatically gets inserted into the housing upon detecting presence of the at least one biological specimen and upon completion of a pre-defined interval of time.

5. The portable medical diagnostic device as claimed in claim 1, wherein the one or more sensors (110) are selected from any one or a combination of a nanosensor and biological sensor.

6. The portable medical diagnostic device as claimed in claim 1, wherein the housing encloses a processor or a microcontroller communicably coupled with the one or more sensors to process the one or more determined parameters by the one or more sensors to generate a printable report (114) causing a portable medical diagnostic device printer (112) embedded in the housing to print the printable report.

7. The portable medical diagnostic device as claimed in claim 1, wherein the housing encloses a processor or a microcontroller communicably coupled with the one or more sensors to process the one or more determined parameters by the one or more sensors to generate a printable report, the processor is configured to transmit the printable report to one or more remote devices (116) through Internet of Things (IOT) devices embedded therein.

8. The portable medical diagnostic device as claimed in claim 1, wherein each of the one or more biological specimen collection modules are loaded with one or more chemical compounds.

9. The portable medical diagnostic device as claimed in claim 1, wherein each of the one or more biological specimen collection modules is removable, bio-degradable and replaceable.

10. The portable medical diagnostic device as claimed in claim 1, wherein each of the one or more biological specimen collection modules may be password protected to prevent unauthorized access.

11. The portable medical diagnostic device as claimed in claim 1, wherein one or more biological specimen are selected from any one or a combination of serum samples, virology swab samples, biopsy and necropsy tissue, cerebrospinal fluid, whole blood for polymerase chain reaction (PCR), and urine samples.
